(19) United States
(12) Patent Application Publication
(10) Pub. No.: US 2021/0068767 A1
(43) Pub. Date: Mar. 11, 2021

(54) BIOLOGICAL INFORMATION MEASURING APPARATUS, BIOLOGICAL INFORMATION MEASURING SYSTEM, AND NON-TRANSITORY COMPUTER READABLE MEDIUM

(71) Applicant: FUJI XEROX CO., LTD., Tokyo (JP)

(72) Inventors: Tadashi SUTO, Kanagawa (JP); Kiichiro ARIKAWA, Kanagawa (JP)

(73) Assignee: FUJI XEROX CO., LTD., Tokyo (JP)

(21) Appl. No.: 16/742,934

(22) Filed: Jan. 15, 2020

(30) Foreign Application Priority Data
Sep. 5, 2019 (JP) 2019-162018

(51) Int. Cl. A61B 5/00; A61B 5/11

(52) U.S. Cl. CPC A61B 5/741; A61B 5/6803; A61B 5/0077; A61B 5/6843; A61B 5/1116; A61B 5/1103

(57) ABSTRACT

A biological information measuring apparatus includes a processor. The processor is configured to obtain information, and if the obtained information indicates that a condition is currently not suitable for measuring biological information of a living body wearing a body of the apparatus, output a guide to a suitable condition for measuring the biological information of the living body.

### CROSS-REFERENCE TO RELATED APPLICATIONS

This application is on the basis of and claims priority under 35 USC 119 from Japanese Patent Application No. 2019-162018 filed Sep. 5, 2019.

### BACKGROUND

### (i) Technical Field

The present disclosure relates to a biological information measuring apparatus, a biological information measuring system, and a non-transitory computer readable medium.

### (ii) Related Art

Japanese Unexamined Patent Application Publication (Translation of PCT Application) No. 2009-530950 discloses a wearable apparatus.

A device for processing data for the wearable apparatus includes an input unit adapted to receive input data, and means for generating information, referred to as wearing information, which is based on sensor information and indicates a state, referred to as wearing state, in which the wearable apparatus is worn. The device for processing data for the wearable apparatus further includes a processing unit adapted to process input data on the basis of the wearing information, thereby generating output data.

### SUMMARY

Aspects of non-limiting embodiments of the present disclosure relate to a biological information measuring apparatus, a biological information measuring system, and a non-transitory computer readable medium each of which is capable of obtaining correct biological information, compared with a case where the wearing state of a user who wears the apparatus is left as it is.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided a biological information measuring apparatus including a processor. The processor is configured to obtain information, and if the obtained information indicates that a condition is currently not suitable for measuring biological information of a living body wearing a body of the apparatus, output a guide to a suitable condition for measuring the biological information of the living body.

### BRIEF DESCRIPTION OF THE DRAWINGS

An exemplary embodiment of the present disclosure will be described in detail on the basis of the following figures, wherein:

FIG. 1 illustrates a biological information measuring system according to the exemplary embodiment;

FIG. 2 illustrates a state in which a user wears a biological information measuring apparatus according to the exemplary embodiment;

FIG. 3 is a sectional view taken along line III-III in FIG. 2;

FIG. 4 is a block diagram illustrating an example of the hardware configuration of the biological information measuring system according to the exemplary embodiment;

FIG. 5 is a flowchart illustrating an example of a measurement-condition correcting process according to the exemplary embodiment;

FIG. 6 is a flowchart illustrating an example of an initial process according to the exemplary embodiment;

FIG. 7 illustrates an operation performed in the initial process according to the exemplary embodiment;

FIG. 8 illustrates an operation performed after the operation illustrated in FIG. 7;

FIG. 9 illustrates an operation performed after the operation illustrated in FIG. 8;

FIG. 10 is a flowchart illustrating an example of an external-apparatus utilizing process according to the exemplary embodiment;

FIG. 11 illustrates an operation performed in the external-apparatus utilizing process according to the exemplary embodiment;

FIG. 12 illustrates an operation performed in the external-apparatus utilizing process according to the exemplary embodiment;

FIG. 13 illustrates an operation performed after the operation illustrated in FIG. 12;

FIG. 14 is a flowchart illustrating an example of a biological-state utilizing process according to the exemplary embodiment;

FIG. 15 is a flowchart illustrating an example of a contact-state determining process according to the exemplary embodiment;

FIG. 16 is a graph illustrating a waveform obtained in the contact-state determining process;

FIG. 17 illustrates an operation performed in the contact-state determining process according to the exemplary embodiment;

FIG. 18 is a flowchart illustrating an example of a blink determining process according to the exemplary embodiment;

FIG. 19 is a graph illustrating a waveform obtained in the blink determining process;

FIG. 20 illustrates an operation performed in the blink determining process according to the exemplary embodiment;

FIG. 21 is a flowchart illustrating an example of a mouth-movement determining process according to the exemplary embodiment;

FIG. 22 is a graph illustrating a waveform obtained in the mouth-movement determining process according to the exemplary embodiment;

FIG. 23 illustrates an operation performed in the mouth-movement determining process according to the exemplary embodiment;

FIG. 24 is a flowchart illustrating an example of a facial-muscle-movement determining process according to the exemplary embodiment;

FIG. 25 is a graph illustrating a waveform obtained in the facial-muscle-movement determining process;

FIG. 26 illustrates an operation performed in the facial-muscle-movement determining process according to the exemplary embodiment;

FIG. 27 is a flowchart illustrating an example of an internal-sensor utilizing process according to the exemplary embodiment;

FIG. 28 is a flowchart illustrating an example of a wearing-state correcting process (1) according to the exemplary embodiment;

FIG. 29 illustrates an operation performed in the wearing-state correcting process (1) according to the exemplary embodiment;

FIG. 30 illustrates an operation performed after the operation illustrated in FIG. 29;

FIG. 31 is a flowchart illustrating an example of a wearing-state correcting process (2) according to the exemplary embodiment;

FIG. 32 illustrates an operation performed in the wearing-state correcting process (2) according to the exemplary embodiment;

FIG. 33 is a flowchart illustrating an example of a biological-movement determining process according to the exemplary embodiment;

FIG. 34 illustrates an operation performed in the biological-movement determining process according to the exemplary embodiment;

FIG. 35 illustrates an operation performed after the operation illustrated in FIG. 34;

FIG. 36 is a flowchart illustrating an example of a biological-state/internal-sensor utilizing process according to the exemplary embodiment;

FIG. 37 is a flowchart illustrating an example of a removal determining process according to the exemplary embodiment;

FIG. 38 illustrates an operation performed in the removal determining process according to the exemplary embodiment;

FIG. 39 is a flowchart illustrating an example of a movement determining process according to the exemplary embodiment;

FIG. 40 illustrates an operation performed in the movement determining process according to the exemplary embodiment; and

FIG. 41 illustrates an operation performed after the operation illustrated in FIG. 40.

### DETAILED DESCRIPTION

Hereafter, an exemplary embodiment will be described with reference to the drawings.

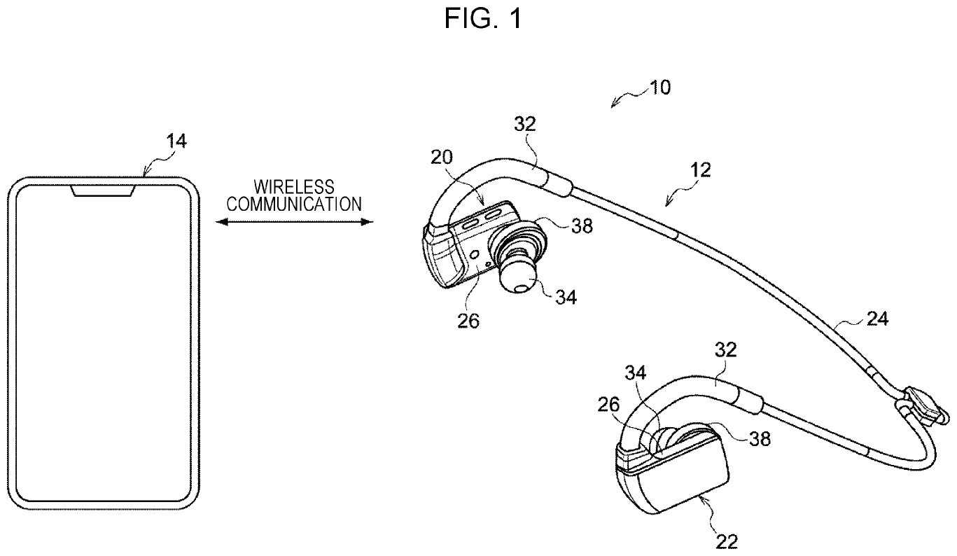

FIG. 1 illustrates a biological information measuring system 10 according to the present exemplary embodiment. The biological information measuring system 10 includes a biological information measuring apparatus 12 and an external apparatus 14.

### External Apparatus

The external apparatus 14 is an apparatus having a detection function and a function of transmitting detected data. Examples of the external apparatus 14 include a personal computer, a smartphone, a tablet information processing apparatus, a measuring instrument such as a contour scanner for detecting an outline, and a terminal such as a personal digital assistant (PDA). In the present exemplary embodiment, the external apparatus 14 is, for example, a smartphone.

### Biological Information Measuring Apparatus

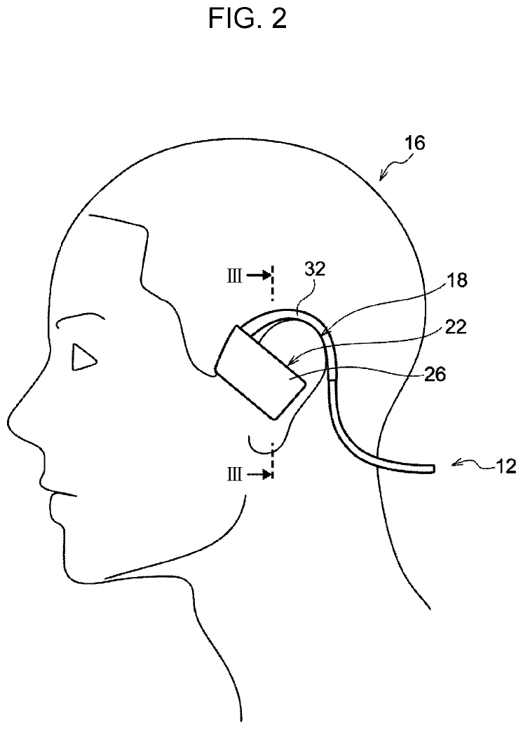

As illustrated in FIG. 2, the biological information measuring apparatus 12 is worn on a living body 16, and obtains biological information indicating a state of the living body 16. Examples of the living body 16 on which the biological information measuring apparatus 12 is worn include animals. Hereafter, an example in which the biological information measuring apparatus 12 is worn on a human, which is an example of an animal, will be described.

Examples of biological information obtained by the biological information measuring apparatus 12 include a pulse, movement of a muscle, and a brain wave. Some items of the biological information, such as movement of a muscle and a brain wave, are examples that generate bioelectricity in a living body. The biological information measuring apparatus 12 according to the present exemplary embodiment measures a brain wave from bioelectricity obtained from the living body.

The biological information measuring apparatus 12 is worn on the head, specifically, ears 18 of the living body 16. As illustrated in FIG. 1, the biological information measuring apparatus 12 includes a right wearing portion 20 to be worn on the right ear, a left wearing portion 22 to be worn on the left ear, and a connection portion 24 that has a wire-like shape and that connects the wearing portions 20 and 22 to each other.

In the present exemplary embodiment, an example in which the biological information measuring apparatus 12 includes the right wearing portion 20, to be worn on the right ear, and the left wearing portion 22, to be worn on the left ear, will be described. However, the biological information measuring apparatus 12 is not limited to this example, and may have only one wearing portion worn on one ear.

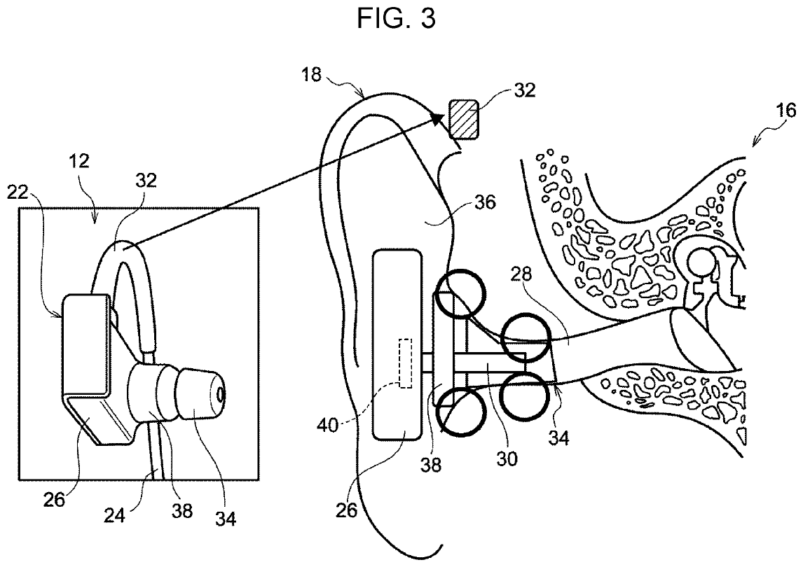

The right wearing portion 20 and the left wearing portion 22 have substantially the same structure. Referring FIG. 3, the left wearing portion 22 will be described as an example.

The left wearing portion 22 includes an apparatus body 26 to which the connection portion 24 is connected and a tubular insertion portion 30 that extends from the apparatus body 26 and that is inserted into an earhole 28. As illustrated in FIGS. 2 and 3, the apparatus body 26 has a laterally elongated rectangular shape. A part of the connection portion 24 near the apparatus body 26 is hardened to form a hanger portion 32, which is hooked on an ear.

A first electrode 34, which is inserted into the earhole 28 and in contact with an inner surface of the earhole, is replaceably attached to a distal end of the insertion portion 30. The first electrode 34 is an earpiece made of an electroconductive rubber. A second electrode 38, which is in contact with an auricle 36, is replaceably attached to a proximal end of the insertion portion 30. The second electrode 38 is a ring-shaped member made of an electroconductive rubber. The circles in FIG. 3 indicate contact points between the electrodes 34 and 38 and the living body 16.

A driver 40, which converts an electric signal into vibration, is provided in the apparatus body 26. The driver 40 reproduces music or sound and outputs the music or sound to the earhole 28 from a hole in the insertion portion 30.

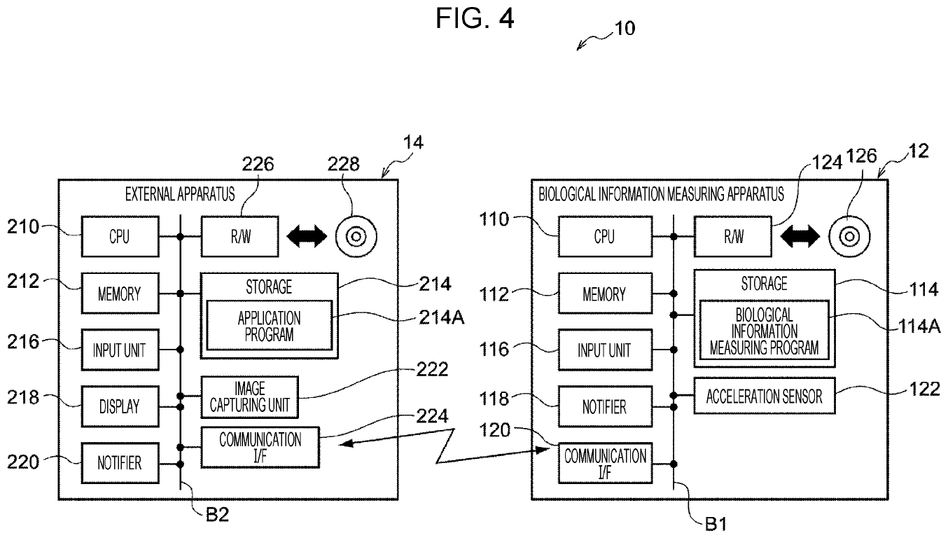

FIG. 4 is a block diagram illustrating an example of the hardware configuration of the biological information measuring system 10. The biological information measuring apparatus 12 and the external apparatus 14, which constitute the biological information measuring system 10, are connected so as to be accessible to each other through communication. Examples of communication include wired communication and wireless communication, such as radio communication or optical communication.

### Hardware Configuration of Biological Information Measuring Apparatus

The biological information measuring apparatus 12 includes a central processing unit (CPU) 110 that serves as a controller and a processor, a memory 112 as a temporary storage area, a non-volatile storage 114, an input unit 116, a notifier 118 including the driver 40, a communication interface (I/F) 120 for performing communication with the external apparatus 14, a three-axis acceleration sensor 122 provided in the apparatus body 26, and a medium reader/writer (R/W) 124 as an example of a device for performing program input.

The CPU 110, the memory 112, the storage 114, the input unit 116, the notifier 118, the communication I/F 120, the acceleration sensor 122, and the medium reader/writer 124 are connected to each other via a bus B1. The medium reader/writer 124 reads information from a recording medium 126 and writes information in the recording medium 126.

A push button switch (not shown) is connected to the input unit 116, and the input unit 116 activates the biological information measuring apparatus 12 when the switch is operated. The electrodes 34 and 38 of the wearing portions 20 and 22 are connected to the input unit 116, and the input unit 116 obtains the potential difference between the electrodes 34 and 38 as a voltage. The input unit 116 amplifies the voltage obtained from the wearing portions 20 and 22 by using a differential amplifier circuit. The input unit 116 sends the potential difference, in which noises simultaneously generated in the electrodes 34 and 38 of the wearing portions 20 and 22 are cancelled out each other, to the CPU 110 as biological information.

The storage 114 may be a hard disk drive (HDD), a solid state drive (SSD), a flash memory, or the like. In the recording medium 126, which serves as a storage, a biological information measuring program 114A for operating the biological information measuring apparatus 12 is stored.

The biological information measuring program 114A is read from the recording medium 126, which is set in the medium reader/writer 124, and is stored in the storage 114. The biological information measuring program 114A may be downloaded via a network.

The CPU 110 reads the biological information measuring program 114A from the storage 114, develops the program 114A in the memory 112, and sequentially executes processes written in the biological information measuring program 114A, thereby serving as a processor and a controller. The biological information measuring apparatus 12 operates as the CPU 110 runs in accordance with the biological information measuring program 114A.

The memory 112 stores the following values (described below) beforehand: a reference range, an allowable angular range, a contact determination threshold, a contact determination period, a blink determination threshold, and a mouth-movement determination threshold.

### Hardware Configuration of External Apparatus

The external apparatus 14 includes a CPU 210, a memory 212 as a temporary storage area, a non-volatile storage 214, an input unit 216 such as a touch panel or a switch, and a display 218 such as a liquid crystal display.

The external apparatus 14 includes a notifier 220 that outputs sound data, an image capturing unit 222 such as a camera for capturing an image, a communication interface (I/F) 224 for performing communication with the biological information measuring apparatus 12, and a medium reader/writer 226.

The CPU 210, the memory 212, the storage 214, the input unit 216, the display 218, the notifier 220, the image capturing unit 222, the communication I/F 224, and the medium reader/writer 226 are connected to each other via a bus B2. The medium reader/writer 226, for example, reads information from a recording medium 228 and writes information in the recording medium 228.

The storage 214 may be a HDD, a SSD, a flash memory, or the like. In the recording medium 228, which serves as a storage, an application program 214A for capturing an image and transmitting captured image data to the biological information measuring apparatus 12 is stored.

The application program 214A is read from the recording medium 228 set in the medium reader/writer 226 and is stored in the storage 214. The application program 214A may be downloaded via a network.

The CPU 210 reads the application program 214A from the storage 214, develops the program 214A in the memory 212, and sequentially executes processes written in the application program 214A.

### Description of Operation

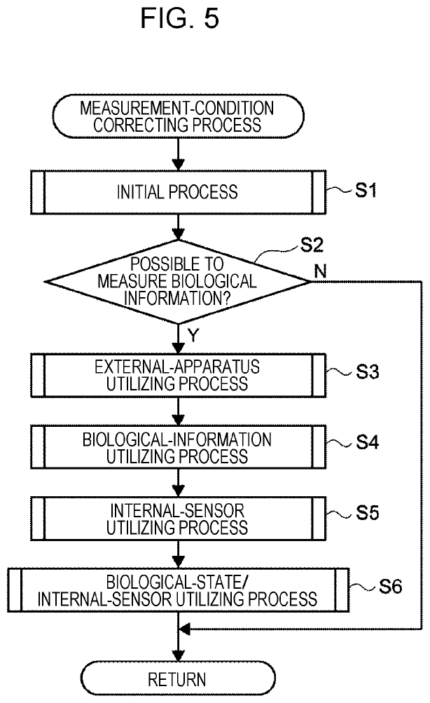

Next, referring to FIG. 5, the operation of the biological information measuring system 10 according to the present exemplary embodiment will be described while focusing on the biological information measuring apparatus 12.

When the CPU 110 of the biological information measuring apparatus 12 executes the biological information measuring program 114A and a measurement-condition correcting process is called from the main routine, the CPU 110 performs an initial process (S1). Then, the CPU 110 determines whether it is possible to measure biological information on the basis of whether the potential difference between the first electrode 34 and the second electrode 38 is input from the input unit 116 (S2).

If it is determined in step S2 that it is not possible to measure biological information, the process returns to the main routine, and notification of an error or the like is performed. If it is determined in step S2 that it is possible to measure biological information, an external-apparatus utilizing process (S3), a biological-state utilizing process (S4), an internal-sensor utilizing process (S5), and a biological-state/internal-sensor utilizing process (S6) are sequentially performed. Then, the process returns to the main routine.

In the present exemplary embodiment, the internal-sensor utilizing process (S5), which uses the acceleration sensor 122, is performed in the measurement-condition correcting process. However, this is not a limitation. For example, depending on a switch input, the internal-sensor utilizing process need not be performed.

### Initial Process

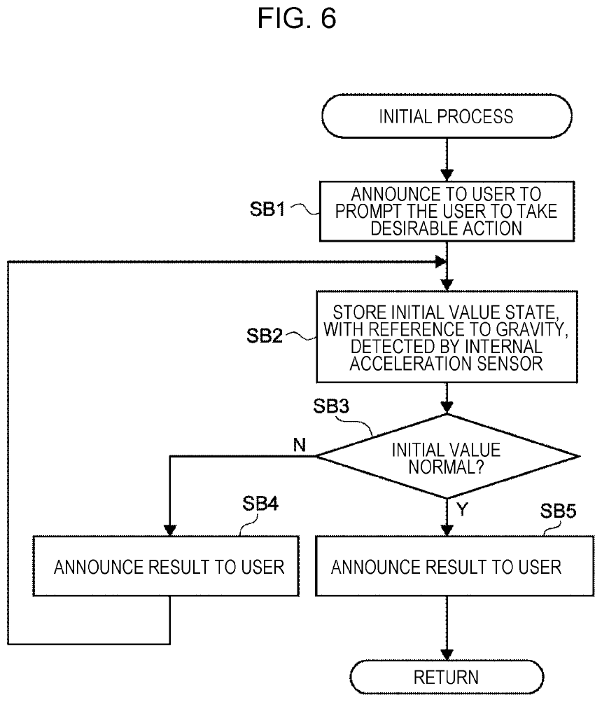

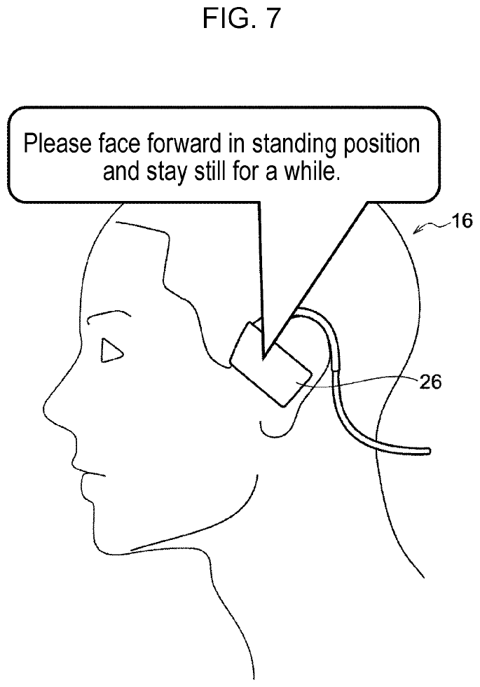

As illustrated in FIG. 6, in the initial process, sound data is output to the driver 40 of the notifier 118, and the driver 40 announces to a user, who is the living body 16 wearing the biological information measuring apparatus 12, to guide the user to take a desirable action (SB1). To be specific, as illustrated in FIG. 7, a sound “Please face forward in standing position and stay still for a while.” is output to the user.

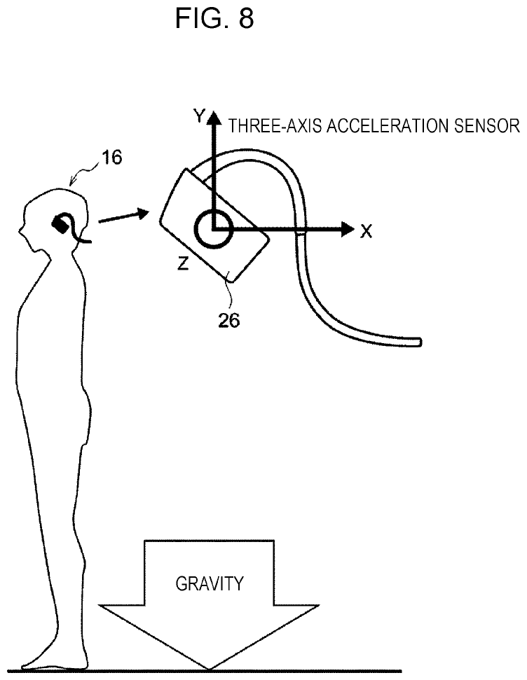

Then, as illustrated in FIG. 8, input data from the acceleration sensor 122, which uses gravity as a reference, is stored in a memory as an initial value (SB2). The acceleration sensor 122 is a three-axis acceleration sensor that detects accelerations in the X direction (the horizontal direction in a standing state), the Y direction (the vertical direction in the standing state), and the Z direction (the left-right direction in the standing state), which are perpendicular to each other. The acceleration sensor 122, which is a sensor for detecting a posture, may be an acceleration sensor having two or more axes.

Next, whether the obtained initial value is normal is determined on the basis of, for example, whether the input data from the acceleration sensor 122 is within a reference range stored in the memory 112 beforehand (SB3).

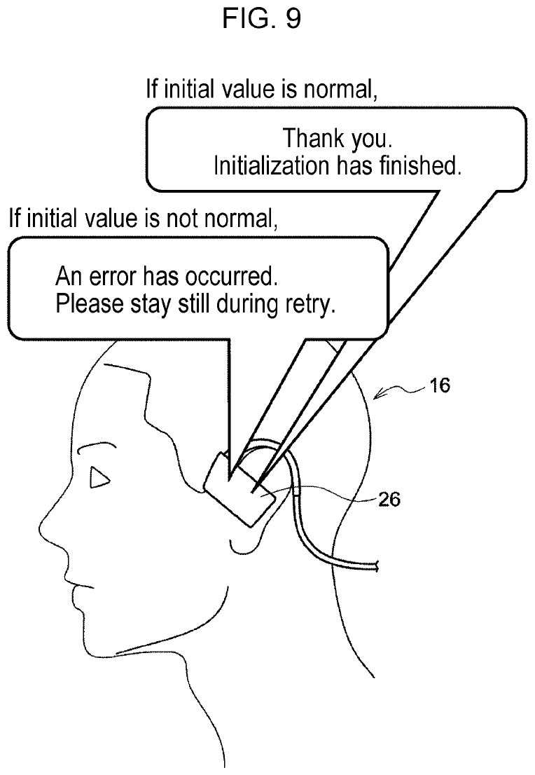

If it is determined in step SB3 that the initial value is not normal, it is determined that an error has occurred. As illustrated in FIG. 9, for example, the driver 40 outputs a sound “An error has occurred. Please stay still during retry.” (SB4), and the process proceeds to step SB2.

If it is determined that the initial value is normal in step SB3, it is determined that the initial process has normally finished. Then, for example, the driver 40 outputs a sound “Thank you. Initialization has finished.” (SB5), and the process returns to the measurement-condition correcting process, which is the called routine.

In the measurement-condition correcting process, as illustrated in FIG. 5, whether it is possible to measure biological information is determined through the aforementioned procedure (S2). If it is determined that it is possible to measure biological information, the external-apparatus utilizing process is performed (S3).

Thus, steps S3 to S6 are performed if the apparatus body 26 can measure biological information.

### External-Apparatus Utilizing Process

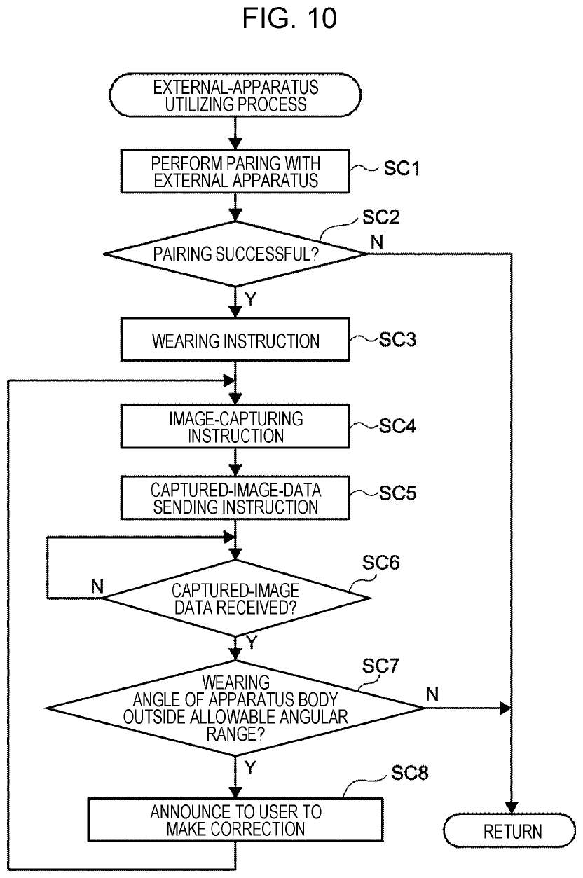

As illustrated in FIG. 10, in the external-apparatus utilizing process, a pairing operation for enabling communication with the external apparatus 14 is performed (SC1). If it is determined that communication with the external apparatus 14 is not possible (SC2), the process returns to the measurement-condition correcting process, which is the called routine. If the communication is possible and the pairing operation succeeds in step SC2, for example, the driver 40 outputs a sound “Please wear the biological information measuring apparatus at the correct position.” to perform a wearing instruction (SC3).

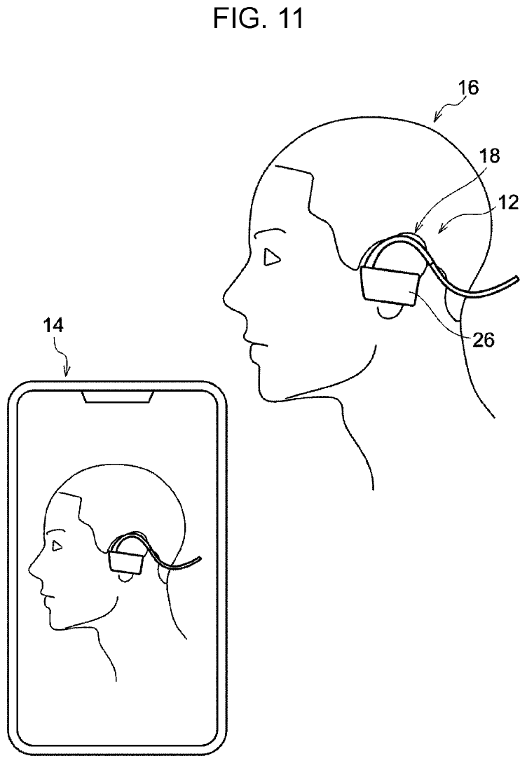

Moreover, for example, the driver 40 outputs a sound “Please capture an image of the wearing state by using an external apparatus.” to perform an image-capturing instruction (SC4), and to guide a user to capture the image by using the external apparatus 14 as illustrated in FIG. 11. Then, for example, the driver 40 outputs a sound “Please send the captured image data.” to instruct the user to send the captured image data (SC5).

Then, the process waits until the communication I/F 120 receives the captured image data (SC6). Thus, the CPU 110 obtains, from the external apparatus 14, information for determining whether the current state enables the apparatus body 26 to measure biological information and the current condition is suitable for measuring the biological information.

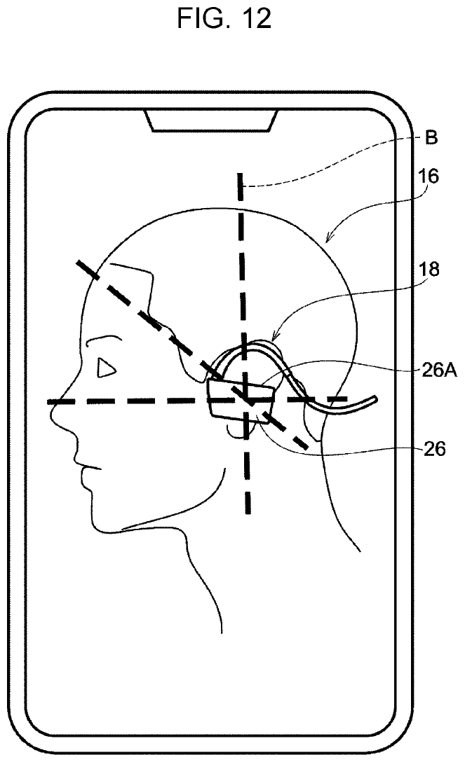

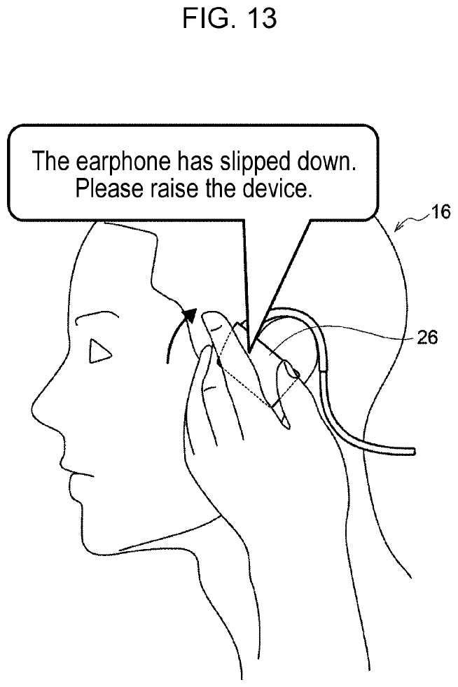

Next, as illustrated in FIG. 12, for example, an upper edge 26A of the apparatus body 26 is detected from the received captured image data, and whether the angle between a vertical line B and the upper edge 26A is within an allowable angular range, which is stored in the memory beforehand, is determined (SC7). If it is determined in step SC7 that the angle is outside the allowable angular range, for example, as illustrated in FIG. 13, the driver 40 outputs a sound “The earphone has slipped down. Please raise the device.” (SC8), and the process proceeds to step SC4.

Thus, if the captured image data indicates that the current condition is not suitable for measuring the biological information, the CPU 110 outputs to the driver 40 guide information that guides a user so that the current condition becomes suitable for measuring the biological information, and the driver 40 outputs the sound.

The wearing instruction (SC3), the image capturing instruction (SC4), the captured-image-data sending instruction (SC5), the determination on the wearing state (SC7), and the operation of outputting the guide information that guides a user to correct the wearing state if the wearing state is inappropriate (SC8) may each be performed by outputting a sound from the notifier 220 of the external apparatus 14 or by displaying a screen on the display 218.

If it is determined in step SC7 that the angle is within the allowable angular range, as illustrated in FIG. 5, the process returns to the measurement-condition correcting process, which is the called routine.

In the present exemplary embodiment, the external-apparatus utilizing process is performed in step S3 of the measurement-condition correcting process. However, this is not a limitation. For example, the external-apparatus utilizing process may be performed as necessary when an error occurs in the biological information or the input from the acceleration sensor 122 during measurement of a brain wave or the like.

### Measurement-Condition Correcting Process

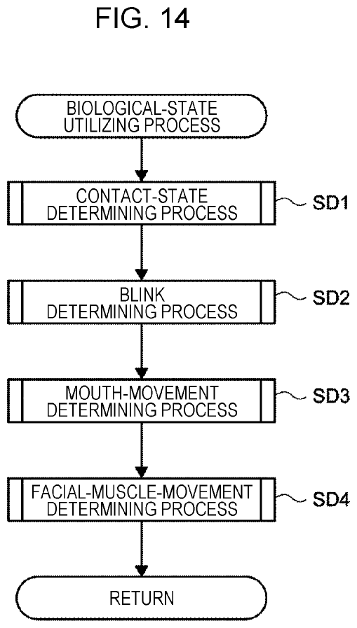

In the measurement-condition correcting process, the biological-state utilizing process is performed (S4). As illustrated in FIG. 14, in the biological-state utilizing process, a contact-state determining process (SD1), a blink determining process (SD2), a mouth-movement determining process (SD3), and a facial-muscle-movement determining process (SD4) are sequentially performed.

### Contact-State Determining Process

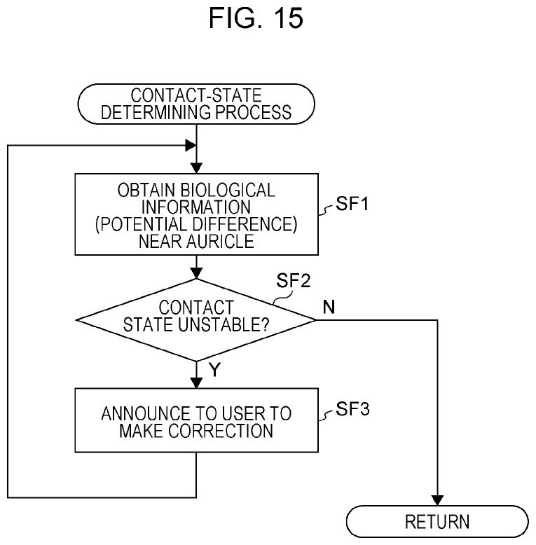

As illustrated in FIG. 15, in the contact-state determining process, biological information near an auricle is obtained by inputting the potential difference from the input unit 116 (SF1), and whether the contact state is unstable is determined from a change in the obtained potential difference (SF2).

In the present exemplary embodiment, whether the contact state is unstable is determined on the basis of a change in the obtained potential difference. However, this is not a limitation. For example, whether the contact state is unstable may be determined on the basis of the difference between voltages input from the electrodes 34 and 38 of the right wearing portion 20 and the left wearing portion 22.

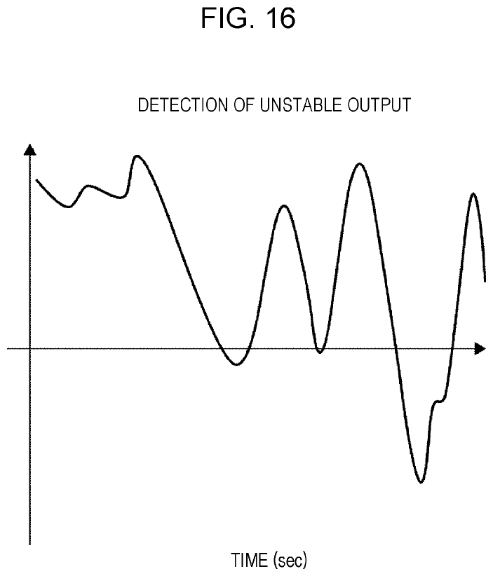

FIG. 16 illustrates change with time in the potential difference detected by the electrodes 34 and 38. It is empirically known that, if the contact state of the electrodes 34 and 38 in contact with the living body 16 is unstable, as illustrated in FIG. 16, a potential difference having a waveform such that the potential difference varies in a large range with an irregular period, compared with the potential difference of a normal brain wave, is detected. Therefore, this range is stored beforehand as a contact determination threshold in the memory 112.

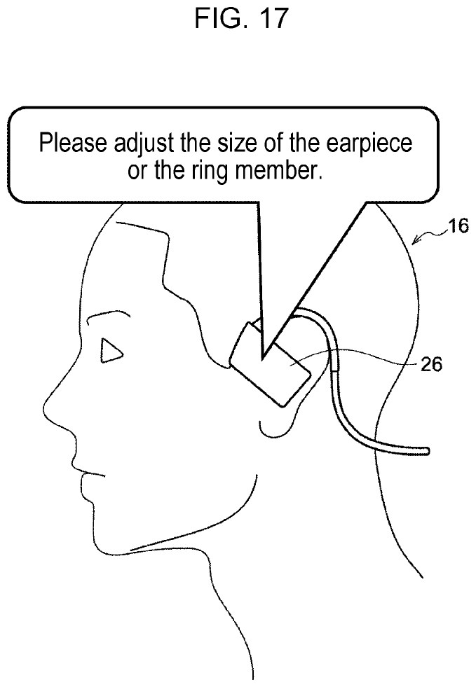

Therefore, if the range of the detected potential difference is larger than the contact determination threshold stored beforehand in the memory 112 and the period of the detected potential difference is larger than a contact determination period stored beforehand in the memory 112, it is determined that the contact state is unstable. Then, as illustrated in FIG. 17, for example, the driver 40 outputs a sound “Please adjust the size of the earpiece or the ring member.” (SF3), and the process proceeds to step SF1.

Thus, guide information that guides a user to adjust the size of each of the electrodes 34 and 38, which are in contact with the ear 18 in the state in which the biological information measuring apparatus 12 is worn on the ear 18, is output on the basis of the contact state.

If it is determined in step SF2 that the contact state is stable on the basis of the change in the obtained potential difference, as illustrated in FIG. 14, the process returns to the biological-state utilizing process, which is the called routine, and the blink determining process is performed (SD2).

### Blink Determining Process

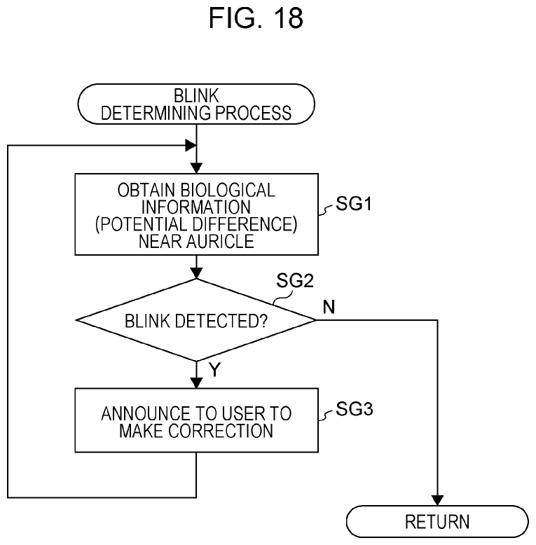

As illustrated in FIG. 18, in the blink determining process, biological information near an auricle is obtained from the potential difference from the input unit 116 (SG1), and whether a blink is detected is determined on the basis of a change in the obtained potential difference (SG2).

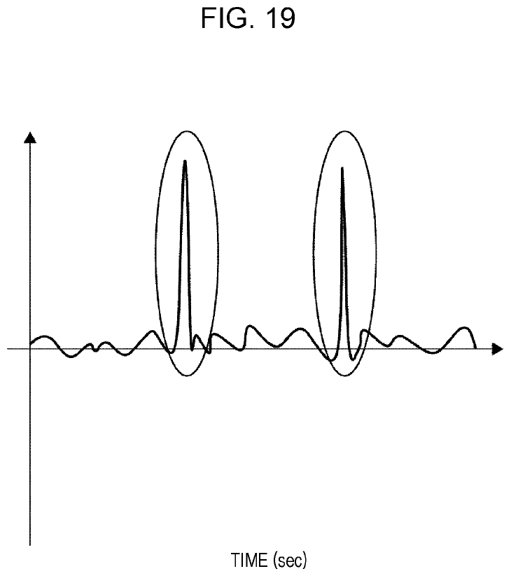

FIG. 19 illustrates change with time in the potential difference input from the input unit 116. When a user blinks, as shown in the ellipses in FIG. 19, a potential difference smaller than 50 μV is detected. The range of the magnitude of the potential difference detected when a user blinks is empirically known, and the range is stored beforehand as a blink determination threshold in the memory 112.

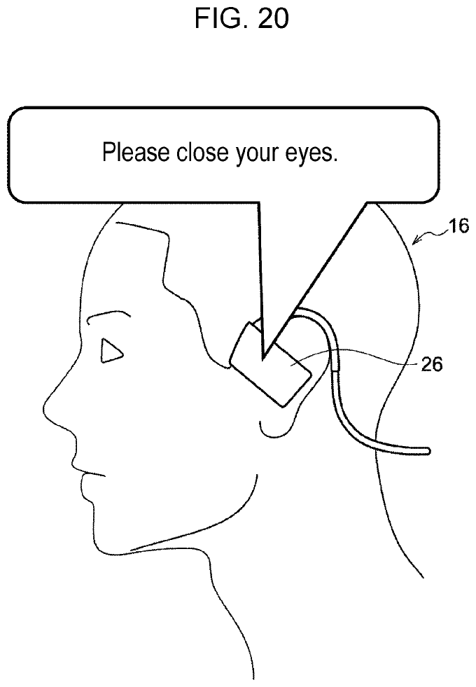

Then, whether a user blinks is detected on the basis of whether the potential difference input from the input unit 116 is within the range indicated by the blink determination threshold stored in the memory 112. If a blink is detected, as illustrated in FIG. 20, for example, the driver 40 outputs a sound “Please close your eyes.” (SG3), and the process proceeds to step SG1.

Thus, guide information that guides a user not to blink is output on the basis of the state of the living body 16 indicated by the potential difference.

If it is determined in step SG2 that a blink is not detected, as illustrated in FIG. 14, the process returns to the biological-state utilizing process, which is the called routine, and the mouth-movement determining process is performed (SD3).

### Mouth-Movement Determining Process

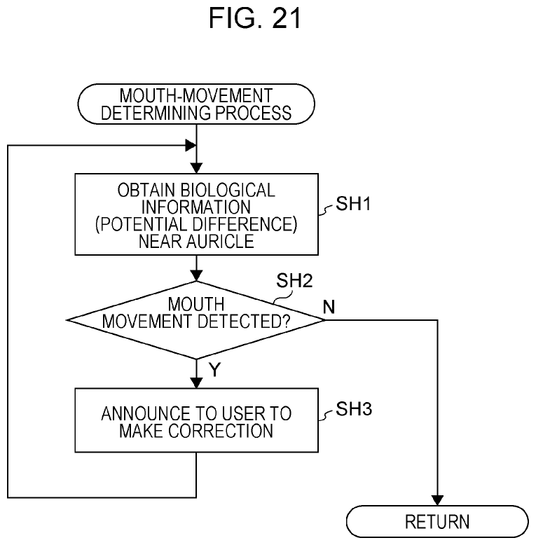

As illustrated in FIG. 21, in the mouth-movement determining process, biological information near an auricle is obtained from the potential difference from the input unit 116 (SH1), and whether movement of the mouth is detected is determined on the basis of a change in the obtained potential difference (SH2).

Here, the term “mouth” has meanings of a mouth and a throat, and may be paraphrased as “a mouth or a throat” or “a mouth and a throat”. Then, movement of the mouth and movement of the throat are detected.

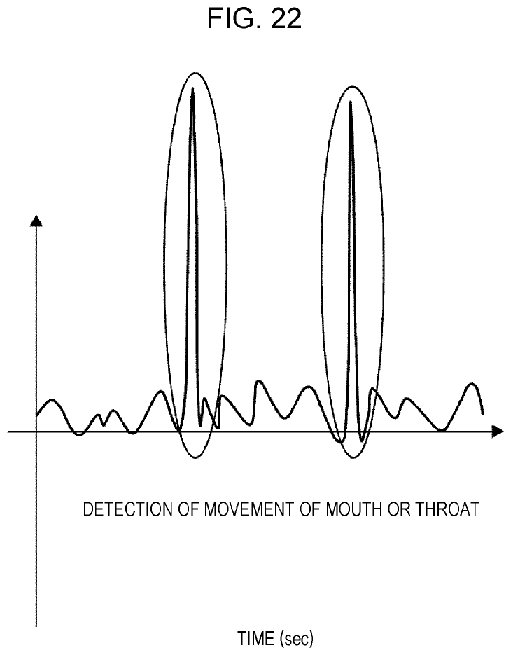

FIG. 22 illustrates change with time in the potential difference input from the input unit 116. When a user moves the mouth, as shown in the ellipses in FIG. 22, a potential difference larger than 50 μV is detected. The potential difference at this time is larger than the potential difference when the user blinks. The range of the magnitude of the potential difference detected when a user moves the mouth is empirically known, and the range is stored beforehand as a mouth-movement determination threshold in the memory 112.

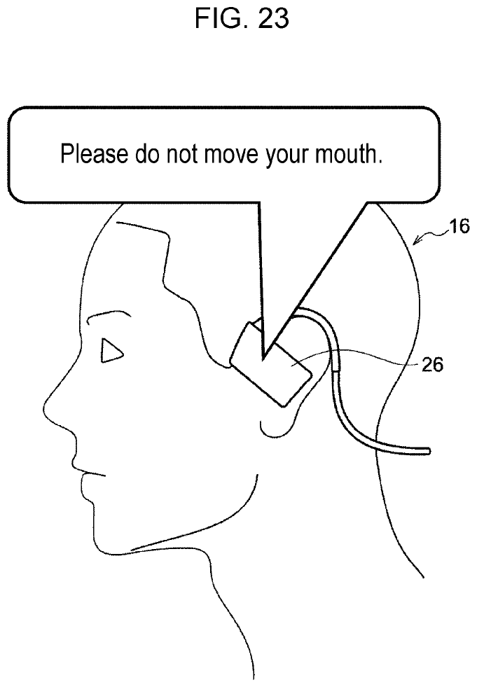

Then, whether a user moves the mouth is detected on the basis of whether the potential difference input from the input unit 116 is within the range indicated by the mouth-movement determination threshold stored in the memory 112. If movement of the mouth is detected, as illustrated in FIG. 23, for example, the driver 40 outputs a sound “Please do not move your mouth.” (SH3), and the process proceeds to step SH1.

Thus, guide information that guides the living body 16 to suppress movement of the mouth is output on the basis of the state of the living body 16 indicated by the potential difference.

If it is determined in step SH2 that movement of the mouth is not detected, as illustrated in FIG. 14, the process returns to the biological-state utilizing process, which is the called routine, and the facial-muscle-movement determining process is performed (SD4).

### Facial-Muscle-Movement Determining Process

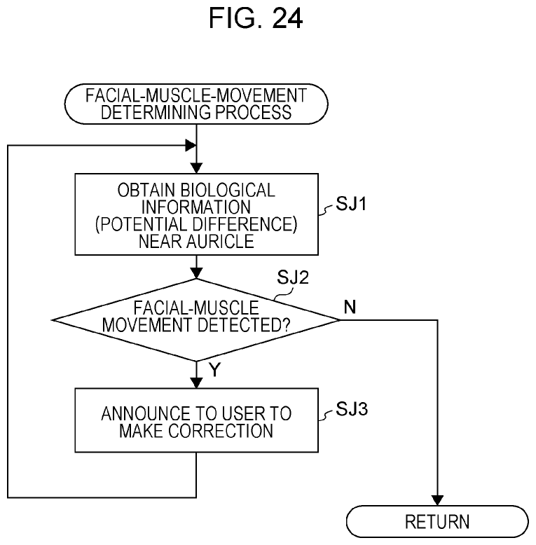

As illustrated in FIG. 24, in the facial-muscle-movement determining process, biological information near an auricle is obtained from the potential difference from the input unit 116 (SJ1), and whether movement of a facial muscle is detected is determined on the basis of a change in the obtained potential difference (SJ2).

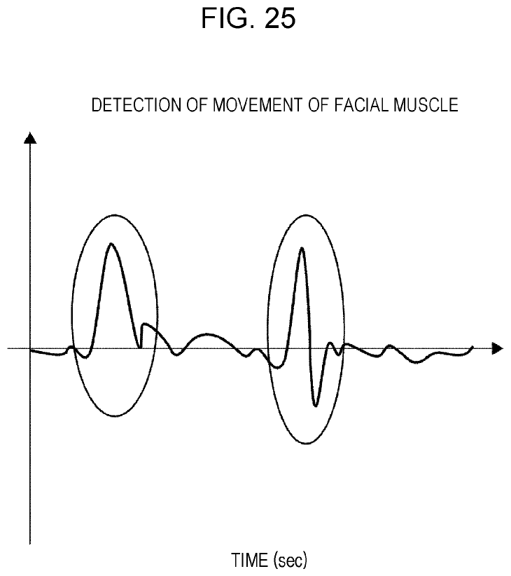

FIG. 25 illustrates change with time of the potential difference input from the input unit 116. When a user moves a facial muscle, as shown in the ellipses in FIG. 25, a potential difference smaller than 50 μV is detected. The width of the waveform of the potential difference at this time is larger than that of a case where the user blinks or a case where the user moves the mouth. The range of the width of the waveform of the potential difference that is detected when a user moves a facial muscle is empirically known, and the range is stored beforehand as a facial muscle determination threshold in the memory 112.

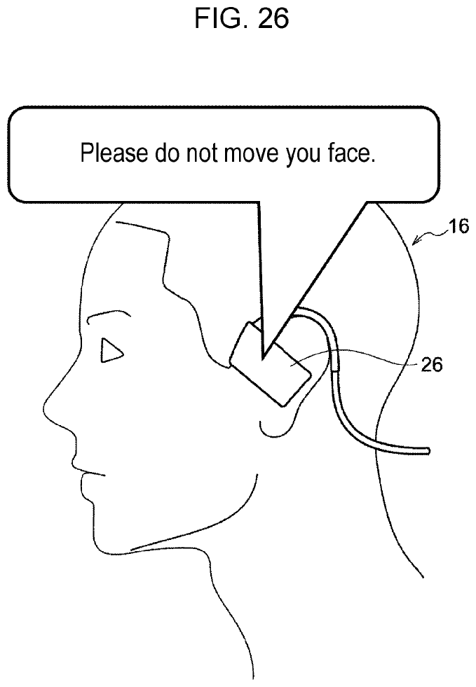

Then, whether a user moves the facial muscle is detected on the basis of whether the width of the waveform of the potential difference input from the input unit 116 is within the range indicated by the facial muscle determination threshold stored in the memory 112. If movement of the facial muscle is detected, as illustrated in FIG. 26, for example, the driver 40 outputs a sound “Please do not move your face.” (SJ3), and the process proceeds to step SJ1.

Thus, guide information that guides the living body 16 to suppress movement of the facial muscle is output on the basis of the state of the living body 16 indicated by the potential difference.

If it is determined in step SJ2 that movement of the facial muscle is not detected, as illustrated in FIG. 14, the process returns to the biological-state utilizing process, which is the called routine. As illustrated in FIG. 5, in the biological-state utilizing process, the process returns to the measurement-condition correcting process, which is the routine that has called the biological-state utilizing process, and the internal-sensor utilizing process is performed (S5).

### Internal-Sensor Utilizing Process

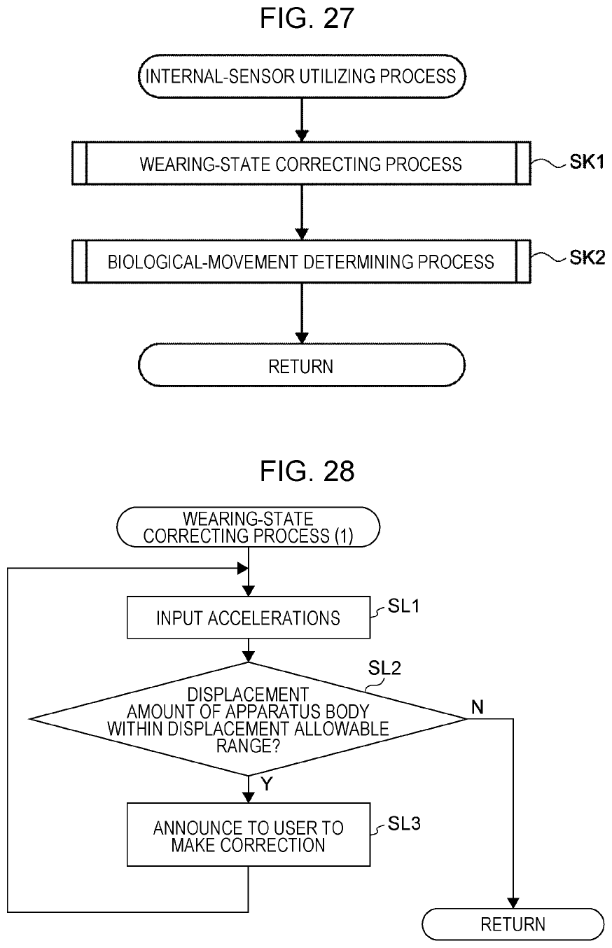

As illustrated in FIG. 27, in the internal-sensor utilizing process, a wearing-state correcting process (SK1) and a biological-movement determining process (SK2) are sequentially performed. As the wearing-state correcting process (SK1), a wearing-state correcting process (1) and a wearing-state correcting process (2) are prepared, and one of the processes (1) and (2) is selectively performed as necessary.

### Wearing-State Correcting Process (1)

As illustrated in FIG. 28, in the wearing-state correcting process (1), accelerations in the three-axis directions detected by the acceleration sensor 122 are input (SL1). By using the accelerations input from the acceleration sensor 122, whether the displacement amount of the apparatus body 26 in the waring state is within an allowable displacement range is determined (SL2).

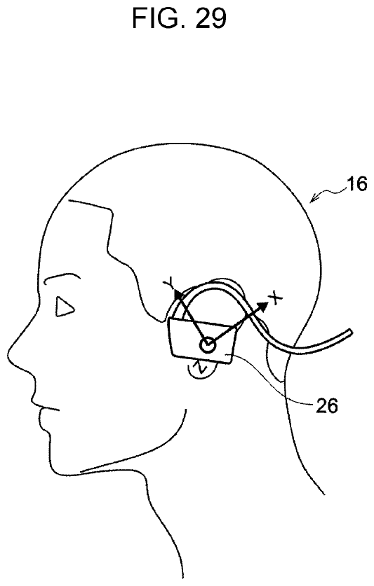

That is, as illustrated in FIG. 29, in a state in which the apparatus body 26 is worn normally, the ranges of the accelerations in the three-axis directions (the X direction, the Y direction, and the Z direction that are perpendicular to each other), which are input from the acceleration sensor 122, are empirical known. The allowable ranges of the accelerations in the three-axis directions are stored beforehand as the displacement-amount determination thresholds in the memory 112.

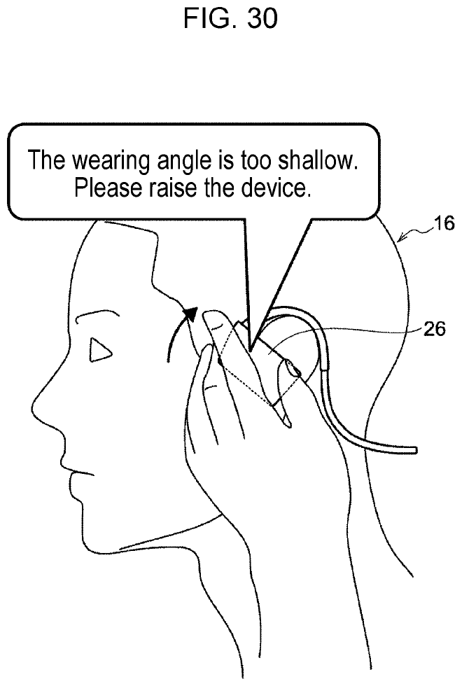

Then, whether displacement the apparatus body 26 in the wearing state is within the displacement allowance range is determined on the basis of whether the accelerations in the three-axis directions, which are input from the acceleration sensor 122, are within the allowable ranges indicated by of the displacement-amount determination thresholds stored in the memory 112. If the displacement amount is outside the allowable displacement range, as illustrated in FIG. 30, for example, the driver 40 outputs a sound “The wearing angle is too shallow. Please raise the device.” (SL3), and the process proceeds to step SL1.

Thus, the acceleration sensor 122, which is provided in the apparatus body 26, is used as a sensor for detecting the posture of the apparatus body 26 in the wearing state. Then, the posture of the apparatus body 26 in the wearing state is detected on the basis of the information obtained by the acceleration sensor 122 provided in the apparatus body 26, and guide information that guide a user to adjust the wearing state of the apparatus body 26 is output.

If it is determined in step SL2 that the displacement amount of the apparatus body 26 in the wearing state is within the allowable displacement range, as illustrated in FIG. 27, the process returns to the internal-sensor utilizing process, which is the called routine.

Here, the internal-sensor utilizing process may call the wearing-state correcting process (2), which uses the initial value stored in in the memory 112 in the initial process, as another example of the wearing-state correcting process.

### Wearing-State Correcting Process (2)

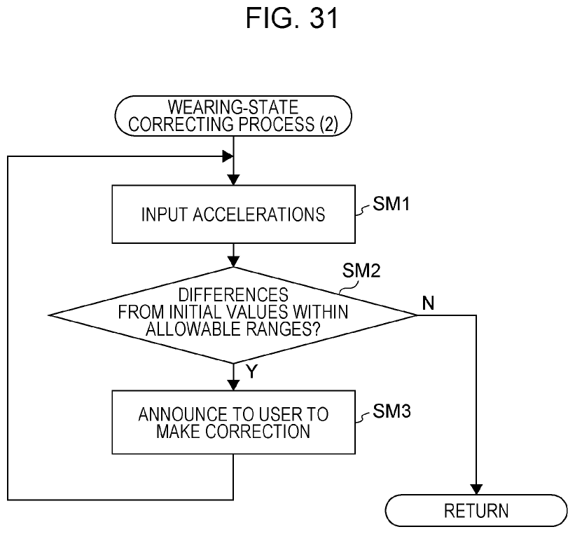

As illustrated in FIG. 31, in the wearing-state correcting process (2), accelerations in the three-axis directions detected by the acceleration sensor 122 are input (SM1), and whether the differences between the accelerations input from the acceleration sensor 122 and the initial values stored in the memory 112 in the initial process are within allowable ranges is determined (SM2).

That is, if the apparatus body 26, which is worn on a user, becomes displaced due to physical exercise or the like, differences between the accelerations input from the acceleration sensor 122 and the initial values obtained in the initial process occur. The ranges of the differences such that the differences do not influence measurement of biological information are empirically known, and the ranges are stored beforehand as allowable displacement ranges in the memory 112.

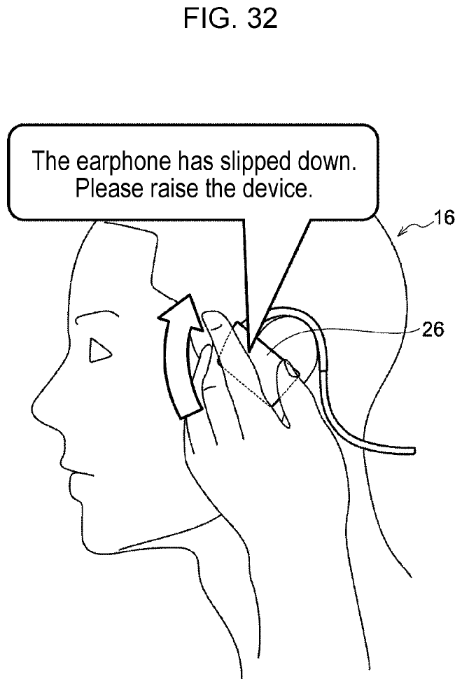

Then, whether the differences between the input accelerations and the initial values are within the allowable displacement ranges stored in the memory 112 is determined. If the differences are outside the allowable displacement ranges, as illustrated in FIG. 32, for example, the driver 40 outputs a sound “The earphone has slipped down. Please raise the device.” (SM3), and the process proceeds to step SM1.

Thus, the acceleration sensor 122, which is provided in the apparatus body 26, is used as a sensor for detecting the posture of the apparatus body 26 worn by the user. Then, on the basis of information obtained by the acceleration sensor 122 of the apparatus body 26, the posture of the apparatus body 26 in the wearing state is detected, and guide information that guides a user to adjust the wearing state of the apparatus body 26 is output.

If it is determined in step SM2 that the differences between the input accelerations and the initial values are within the allowable displacement ranges, as illustrated in FIG. 27, the process returns to the internal-sensor utilizing process, which is the called routine, and the biological-movement determining process is performed (SK2).

### Biological-Movement Determining Process

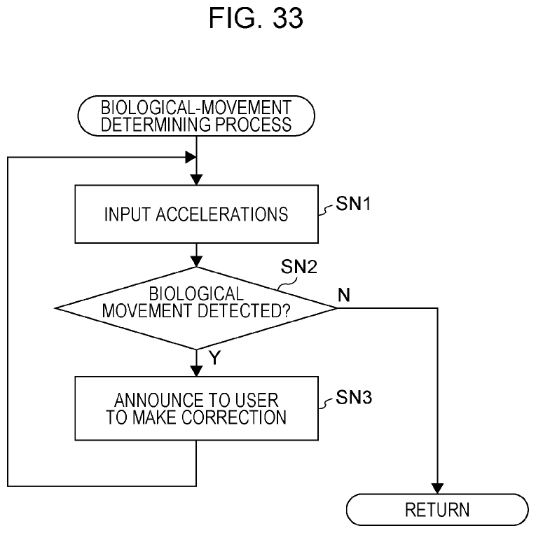

As illustrated in FIG. 33, in the biological-movement determining process, accelerations detected by the acceleration sensor 122 are input (SN1), and whether a user wearing the apparatus body 26 moves is determined (SN2).

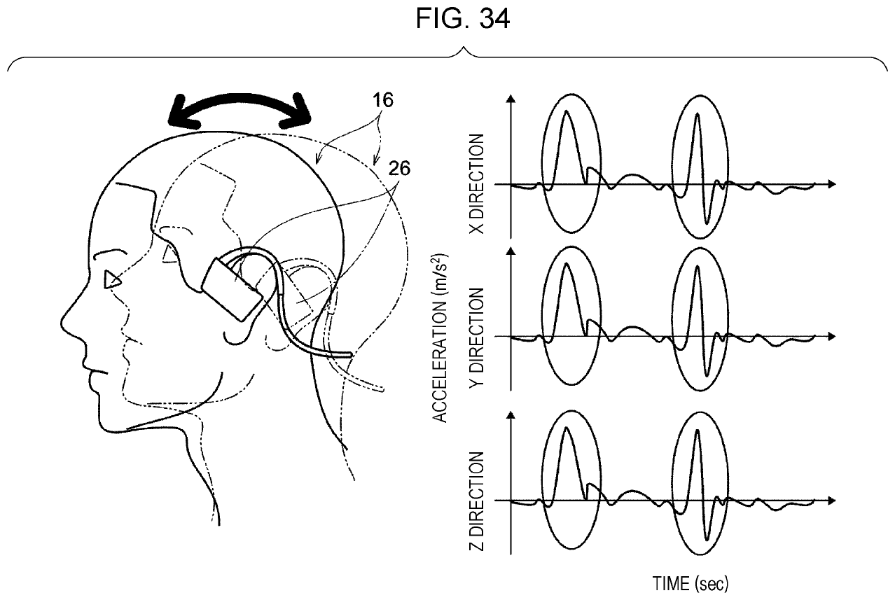

That is, as illustrated in FIG. 34, for example, if the user moves his/her head, the waveforms of the acceleration in the X direction, the acceleration in the Y direction, and the acceleration in the Z direction input from the acceleration sensor 122 change. The ranges of the changes such that the changes do not influence measurement of biological information are empirically known, and the ranges are stored beforehand as allowable change ranges in the memory 112.

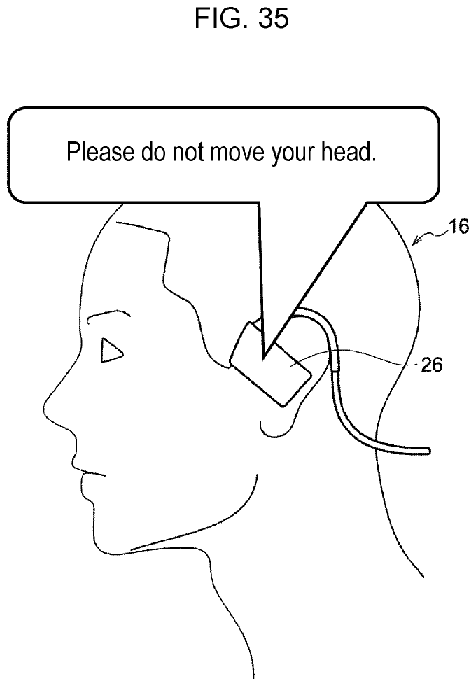

Then, whether changes of the input accelerations in the X, Y, and Z directions are within the allowable change ranges stored in the memory 112 is determined. If the changes are outside the allowable change ranges, as illustrated in FIG. 35, for example, the driver 40 outputs a sound “Please do not move your head.” (SN3), and the process proceeds to step SN1.

Thus, the acceleration sensor 122, which is provided in the apparatus body 26, is used as a sensor for detecting movement of the apparatus body 26. Then, on the basis of information obtained by the acceleration sensor 122 provided in the apparatus body 26, movement of the apparatus body 26 in the wearing state is detected, and guide information that guides the living body 16 to suppress movement of the living body 16 is output.

If it is determined in step SN2 that the input accelerations in the X, Y, and Z directions are within the allowable change ranges, as illustrated in FIG. 27, the process returns to the internal-sensor utilizing process, which is the called routine. As illustrated in FIG. 5, in the internal-sensor utilizing process, the process returns to the measurement-condition correcting process, from which the internal-sensor utilizing process is called, and the biological-state/internal-sensor utilizing process is performed (S6).

### Biological-State/Internal-Sensor Utilizing Process

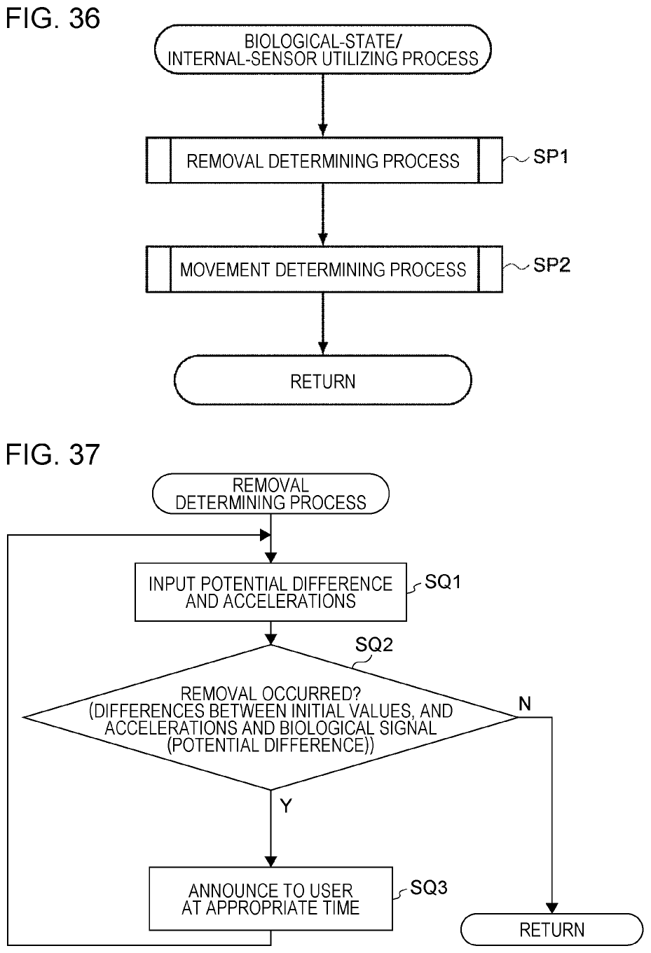

As illustrated in FIG. 36, in the biological-state/internal-sensor utilizing process, a removal determining process (SP1) and a movement determining process (SP2) are sequentially performed.

### Removal Determining Process

As illustrated in FIG. 37, in the removal determining process, biological information is input from the input unit 116 as a potential difference, and accelerations detected by the acceleration sensor 122 are input (SQ1). Then, whether the apparatus body 26 is removed is determined by comparing the input accelerations with the initial values stored in the memory 112 and by measuring a change in the potential difference (SQ2).

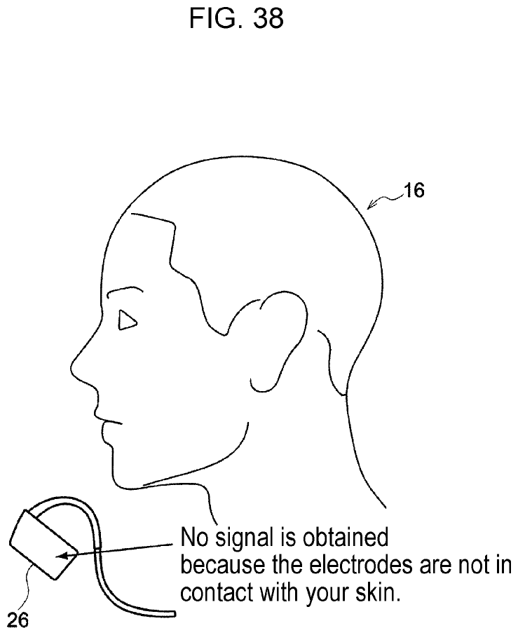

That is, when the apparatus body 26 is removed as illustrated in FIG. 38, the accelerations from the acceleration sensor 122 considerably change compared with the initial values.

At this time, if the accelerations input from both of the acceleration sensors 122 of the right wearing portion 20 worn on the right ear and the left wearing portion 22 worn on the left ear considerably change, it can be determined that the wearing portions 20 and 22 are removed. If the accelerations input from the acceleration sensor 122 of only one of the wearing portions 20 and 22 considerably change, it can be determined that the apparatus body 26 is temporarily removed from one ear.

Because the electrodes 34 and 38 are no longer in contact with the living body 16 when the apparatus body 26 is removed, it is not possible to obtain the potential difference, which is a biological signal.

If both of the wearing portions 20 and 22 are removed, the input potential difference approaches “0”. If only one of the wearing portions 20 and 22 is removed, the potential difference is considerably disturbed. Thus, it can be determined that the apparatus body 26 is temporarily removed from one ear.

Then, on the basis of these conditions, guide information is output at an appropriate timing.

To be specific, if it is determined that the apparatus body 26 is temporarily removed from only one ear as described above, a sound is not output for a predetermined time, and, after the predetermined time has elapsed, for example, the driver 40 outputs a sound “Please wear the apparatus body.” (SQ3), and the process proceeds to step SQ1. The predetermined time may be, for example, 3 minutes.

In step SQ3, if determination based on the accelerations and determination based on the potential difference differ from each other, for example, the driver 40 may output a sound indicating an error.

If it is determined in step SQ2 that the apparatus body 26 is worn, as illustrated in FIG. 36, the process returns to the biological-state/internal-sensor utilizing process, which is the called routine, and the movement determining process is performed (SP2).

### Movement Determining Process

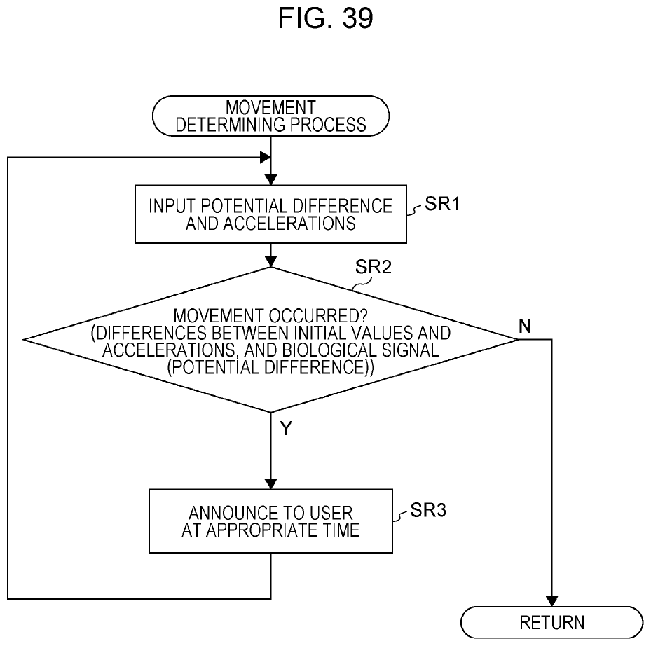

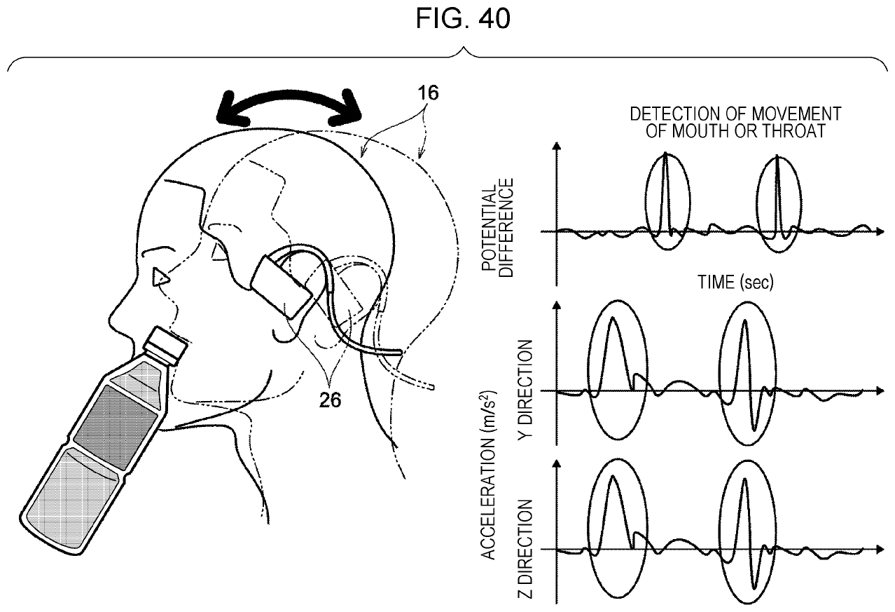

As illustrated in FIG. 39, in the movement determining process, biological information is input as a potential difference, and accelerations detected by the acceleration sensor 122 are input (SR1). Then, whether a user moves is determined by comparing the input accelerations with the initial values stored in the memory 112 and by measuring a change in the potential difference (SR2).

That is, as illustrated in FIG. 40, when the user has a drink, as an example of movement of a user, among the accelerations from the acceleration sensor 122, for example, the acceleration in the X direction and the acceleration in the Y direction change considerably as shown in the ellipses. Moreover, because the mouth also moves, the potential difference changes as shown in the ellipses immediately after the accelerations change.

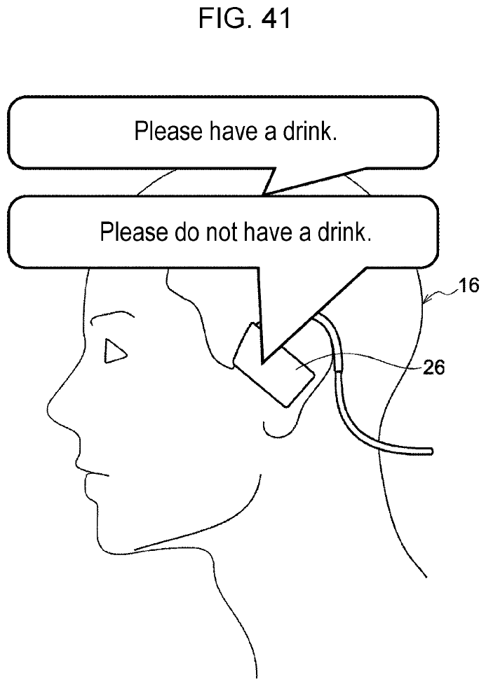

Therefore, for example, if the potential difference changes immediately after the acceleration in the X direction and the acceleration in the Y direction considerably change, it is determined that the user has moved to have a drink. Then, as illustrated in FIG. 41, for example, the driver 40 outputs a sound “Please do not have a drink.” (SR3), and the process proceeds to step SR1.

Here, for example, if it is determined that a user has not had a drink for a predetermined time, it is possible to guide the user to hydrate periodically by causing the driver 40 to output a sound “Please have a drink (SR3). Also in a case of measuring a brain wave of a user after the user has a drink, for example, the driver 40 outputs a sound “Please have a drink.” (SR3).

If it is determined in step SR2 that a user does not move, as illustrated in FIG. 36, the process returns to the main routine via the biological-state/internal-sensor utilizing process and the measurement-condition correcting process, which are called routines.

In the present exemplary embodiment, the driver 40 outputs guide information. However, this is not a limitation. For example, guide information may be sent to the external apparatus 14, and the external apparatus 14 may output the guide information.

A method for outputting guide information is not limited to a method of outputting guide information as a sound. For example, guide information may output as characters, a display, vibration, or the like.

The timing at which guide information is output may be changed in accordance with situations. If the measurement condition is not improved for a predetermined time after guide information is output, for example, the guide information may be continued to be output for three minutes and the guide information may be output again after one hour.

In the embodiment above, the term “processor” refers to hardware in a broad sense. Examples of the processor includes general processors (e.g., CPU: Central Processing Unit), dedicated processors (e.g., graphics processing unit (GPU), application specific integrated circuit (ASIC), field programmable gate array (FPGA), and programmable logic device).

In the embodiment above, the term “processor” is broad enough to encompass one processor or plural processors in collaboration which are located physically apart from each other but may work cooperatively. The order of operations of the processor is not limited to one described in the embodiments above, and may be changed.

In the present exemplary embodiment, all of the processes illustrated in FIG. 5 are performed. However, this is not a limitation. Only some of these processes may be performed. Further alternatively, only some of the subroutines of the processes illustrated in FIG. 5 may be performed.

If it is determined in each the processes that it is not suitable for measure biological information, measurement data obtained during the process may be deleted.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiment s and with the various modifications as are suited to the particular use contemplated. It is intended that the scope of the disclosure be defined by the following claims and their equivalents.

What is claimed is:

1. A biological information measuring apparatus comprising:
a processor configured to
obtain information, and
if the obtained information indicates that a condition is currently not suitable for measuring biological information of a living body wearing a body of the apparatus, output a guide to a suitable condition for measuring the biological information of the living body.

2. The biological information measuring apparatus according to claim 1, wherein the guide is output as a sound.

3. The biological information measuring apparatus according to claim 1, wherein the information is obtained from a state of the living body on which the body of the apparatus is worn.

4. The biological information measuring apparatus according to claim 2, wherein the information is obtained from a state of the living body on which the body of the apparatus is worn.

5. The biological information measuring apparatus according to claim 3,
wherein the guide is output if a blink of the living body is detected from a potential difference generated between a pair of electrodes in contact with the living body, and
wherein the guide directs the living body not to blink.

6. The biological information measuring apparatus according to claim 4,
wherein the guide is output if a blink of the living body is detected from a potential difference generated between a pair of electrodes in contact with the living body, and
wherein the guide directs the living body not to blink.

7. The biological information measuring apparatus according to claim 3,
wherein the guide is output if movement of a mouth is detected from a potential difference generated between a pair of electrodes in contact with the living body, and
wherein the guide directs the living body to suppress movement of the mouth of the living body.

8. The biological information measuring apparatus according to claim 5,
wherein the guide is output if movement of a mouth is detected from a potential difference generated between a pair of electrodes in contact with the living body, and
wherein the guide directs the living body to suppress movement of the mouth of the living body.

9. The biological information measuring apparatus according to claim 3,
wherein the guide is output if movement of a facial muscle is detected from a potential difference generated between a pair of electrodes in contact with the living body, and
wherein the guide directs the living body to suppress movement of the facial muscle of the living body.

10. The biological information measuring apparatus according to claim 3,
wherein the processor detects a contact state of a pair of electrodes from a potential difference generated between the electrodes, the electrodes being in contact with the living body when the body of the apparatus is worn on an ear of the living body,
wherein the guide is output based on the contact state of the pair of electrodes, and
wherein the guide directs the living body to adjust sizes of the electrodes.

11. The biological information measuring apparatus according to claim 1, wherein the information is obtained by using a sensor provided in the body of the apparatus.

12. The biological information measuring apparatus according to claim 2, wherein the information is obtained by using a sensor provided in the body of the apparatus.

13. The biological information measuring apparatus according to claim 11,
wherein the sensor detects a posture of the body of the apparatus, and
wherein the guide directs the living body to adjust a wearing state of the body of the apparatus.

14. The biological information measuring apparatus according to claim 11,
wherein the sensor detects movement of the body of the apparatus, and
wherein the guide directs the living body to suppress movement of the living body.

15. The biological information measuring apparatus according to claim 1, wherein the information is obtained from an external apparatus.

16. The biological information measuring apparatus according to claim 2, wherein the information is obtained from an external apparatus.

17. The biological information measuring apparatus according to claim 15,
wherein the information obtained from the external apparatus is captured image data, the captured image data being data of an image of a wearing state in which the body of the apparatus is worn on the living body,
wherein the guide is output in response to the wearing state being inappropriate, and
wherein the guide directs the living body to correct the wearing state of the body of the apparatus.

18. A biological information measuring system comprising:
the biological information measuring apparatus according to claim 15; and
the external apparatus.

19. A non-transitory computer readable medium storing a program causing a computer to execute a process for measuring biological information, the process comprising:
obtaining information; and
if the obtained information indicates that a condition is currently not suitable for measuring biological information of a living body wearing a body of the apparatus, outputting a guide to a suitable condition for measuring the biological information of the living body.

20. A biological information measuring apparatus comprising:
a body comprising an electrode that obtains biological information by being in contact with a living body; and
a controller configured to:
obtain information, and
if the obtained information indicates that a condition is currently not suitable for measuring biological information of the living body wearing a body of the apparatus, output a guide to a suitable condition for measuring the biological information of the living body.
